(19) United States
(12) Patent Application Publication
(10) Pub. No.: US 2004/0260273 A1
(43) Pub. Date: Dec. 23, 2004

(54) Magnetic surgical instrument system

(76) Inventor: Elaine Y. Wan, Fresh Meadows, NY (US)

Correspondence Address: Leslie Gladstone Restaino, 163 Madison Avenue, P.O. Box 1989, Morristown, NJ, 07962-1989, US

(21) Appl. No.: 10/465,466

(22) Filed: Jun. 18, 2003

(51) Int. Cl. A61B 17/00

(52) U.S. Cl. 606/1

(57) ABSTRACT

The present invention provides a magnetic surgical instrument system for use in transmural surgical operations. The system includes at least two magnetic probes including magnetic components, such as a permanent magnet, an electromagnet, and a metal attracted to a magnet that attracts the magnetic probes to provide therewith a magnetic clamping force for clamping the wall of anatomical structures. The system may also include a driving device, which provides the driving signal to produce electromagnetic force and energy for various embodiments of the present invention, such as RF, microwave, laser, and cryogenic energy. The magnetic surgical system may also be adopted to ablate, cut, stable, and inject anatomical structure tissue clamped between the magnetic probes.

### BACKGROUND OF THE INVENTION

The present invention relates to surgical instruments. Particularly, the invention relates to surgical instrument systems that permit less invasive transmural surgical operations or interventions on anatomical structures having a lumen. Transmural is used herein to denote through the wall of anatomical structures having a lumen. An anatomical structure having a lumen is used herein to denote any part of a body, human or otherwise, and any bodily organ that has a cavity or hollow space associated therewith which is defined by the wall or walls of the part of the body or bodily organ, such as the heart, lungs, bladder, esophagus, stomach, intestines, thoracic cavity, abdominal cavity, blood vessels, etc.

Numerous surgical procedures require invasive techniques to access anatomic structures targeted for transmural surgical operations and also to perform the transmural surgical operations. The Maze procedure for treating atrial fibrillation, for instance, is typically performed with invasive techniques both to gain access to the subject's heart and also to surgically correct the defect causing the atrial fibrillation. The Maze procedure generally entails interrupting the arrhythmia causing electrical impulses to the relevant section of the heart by creating transmural incisions in strategic locations in the atria, which form into scar tissue to permanently block electrical impulses. In order to perform the Maze procedure, a surgeon invasively gains direct access to the heart by dividing and spreading the patient's sternum. The incision and suturing the patient's heart requires that the heart not beat during the procedure, accordingly, a cardiopulmonary bypass is required to supply blood to the patients organ during the Maze procedure. Although, the open surgical Maze procedure is reported to be over 90% effective in treating atrial fibrillation, the procedure is relatively complex lasting up to 8 hours and has a significant recovery period of about 6-8 weeks.

Less invasive catheter ablation techniques have been applied to treat arrhythmias, as discussed in U.S. Pat. No. 5,429,131, entitled “Magnetized Electrode Tip Catheter,” which is hereby incorporated herein by reference, however, the ablation catheters appearing in the art have numerous shortcomings. The energy emitting portion of the ablation catheters appearing in the art, for instance, are generally adopted only to apply energy to one side of a wall of the heart, which, with respect to relatively thick heart walls, may result in excess charring and/or incomplete transmural penetration, which necessarily limits their effectiveness with respect to relatively thick sections of the heart, such as the ventricular heart walls and the muscular portion of the interventricular septum. Additionally, at least with respect to the heart, since the ablation catheters in the art are designed for intraluminal access, the energy supplied by the ablation catheter is applied only to the inner side of the walls of the heart, i.e., the endocardium. Correspondingly, the ablation catheters appearing in the heart have had limited success with respect to treating certain types of atrial fibrillation. There is therefore a need for surgical instruments that provide less invasive techniques for their use in transmural surgical operations without some or all of the shortcomings associated with those in the art.

### SUMMARY OF THE INVENTION

The present invention provides magnetic surgical instrument systems which enable users to perform therewith less invasive surgical operations, such as in connection with the Maze procedure. This is accomplished with a magnetic surgical instrument system that includes at least two magnetic probes, e.g., a first and second magnetic probe, and a driving device. Each magnetic probe has at least one functional component associated therewith, which is a magnetic component. The magnetic component may be a permanent magnet, an electromagnet, and a metal attracted to a magnet, however, at least one of the magnetic components, e.g., of the first magnetic probe, is an electromagnet. The magnetic probes that include electromagnet magnetic components are operatively connected to the driving device, which generally provides the electric current to energize the electromagnet and produce therewith a magnetic clamping force, thereby enabling a user to clamp walls of an anatomical structure between the magnetic probes.

The driving device may provide various functions. In one embodiment, the driving device is adopted to enable a user to selectively energize the electromagnet of the first magnetic probe thereby allowing the magnetic clamping force between the magnetic components to be turned on and off. The driving device may be adopted to provide an adjustable magnetic clamping force, thereby enabling a user to vary the pressure applied to the walls of the anatomical structure. At least one of the magnetic probes, e.g., the first magnetic probe, may include a pressure sensor that supplies data associated with the amount of clamping force produced with the magnetic probes. The data may generally be used to monitor the pressure applied to the wall of the anatomical structure. In one embodiment, the driving device is adopted to actively monitor and adjust the pressure applied to the wall of the anatomical structure.

The surgical instrument may also be adopted to create lesions in the walls of anatomical structure clamped between the magnetic probes. In this instance, at least one of the magnetic probes includes a functional element that is an electrode. The magnetic probe including the electrode is operatively connected to the driving device which supplies energy to the electrode sufficient for a user to create transmural lesions therewith in the walls of the anatomical structure clamped between the magnetic probes. In one embodiment, at least two of the magnetic probes include an electrode. In this instance, these magnetic probes are operatively connected to the driving device which supplies energy to the electrodes sufficient for a user to create transmural lesions therewith in the walls of the anatomical structure clamped between the magnetic probes. The electrodes may provide various types of energy. Accordingly, the energy supplied by the driving devices may be selected from the group consisting of radiofrequency energy, microwave energy, laser energy, cryothermic energy, etc. In one embodiment, the energy supplied to the electrodes is radio frequency energy and one magnetic probe comprises an active electrode of a bipolar electrode pair and another magnetic probe is a return electrode of the bipolar electrode pair. The electrodes may be either monopolar or bipolar (an active and return electrode pair). In either event, the monopolar or bipolar electrode included in each of the magnetic probes provides the ability for users to create transmural lesions from both sides of the walls of the anatomical structure.

The driving device may be adopted to supply an adjustable amount of energy to the electrodes thereby enabling a user to vary the amount of energy to create the transmural lesions. In one embodiment, at least one of the magnetic probes includes a temperature sensor for monitoring and adjusting the temperature at the site of the transmural lesion. In another embodiment, at least two of the magnetic probes include a temperature sensor and the driving device is adopted to enable a user to independently adjust the amount of energy supplied to the electrode of each of the magnetic probes. Alternatively or in addition, the driving device may be adopted to monitor the temperature at the site of the lesion and automatically adjust the energy supplied to each of the magnetic probes. The driving device 210 may also be adopted to monitor the amount of time that energy is applied to the walls of the anatomic structure and may adjust or cutoff the amount of energy supplied to the electrode based on the time that energy is applied to the walls of the anatomic structure.

The surgical instrument may be adopted to staple, cut, incise, or inject the walls of the anatomical structure clamped between the magnetic probes. In one embodiment, the first magnetic probe includes a functional element that is a stapler mechanism containing a staple and capable of driving a staple into the wall of the anatomical structure clamped between the magnetic probes. The force necessary to drive the staple into the wall of the anatomical structure may be provided by the magnetic clamping force between the magnetic probes. The stapler may be a retractable stapler containing a staple therein, which is ejected out of the retractable stapler upon application of the magnetic clamping force on the magnetic probes.

In another embodiment, the first magnetic probe includes a functional element that is a blade capable of incising at least partially through the wall of the anatomical structure clamped between the magnetic probes. The force necessary to incise the wall of the anatomical structure may be provided by the magnetic clamping force between the magnetic probes. The blade may be a retracted blade, which is ejected out of the magnetic probe upon application of the magnetic clamping force on the magnetic probes.

In another embodiment, the first magnetic probe includes a functional element that is at least one needle for injecting substances into the wall of the anatomical structure clamped between the magnetic probes. The force necessary to drive the needle into the wall of the anatomical structure may be provided by the magnetic clamping force between the magnetic probes. The needle or needles may be retracted and ejected out of the magnetic probe upon application of the magnetic clamping force on the magnetic probes.

In another embodiment, the first magnetic probe includes a functional element that is at least one suturing needle and includes a suture receptacle, which provides sutures for the suturing needle, which allows a user therewith to suture walls of an anatomical structure clamped between the magnetic probes.

In one aspect of the present invention, a magnetic surgical instrument system for use in transmural surgical operations is provided which includes at least two magnetic probes and a driving device, where each magnetic probe has a plurality of functional components associated therewith which include a magnetic component and an electrode. The magnetic components generally produce a magnetic clamping force, which allows a user to clamp walls of an anatomical structure between the magnetic probes. Additionally, the electrode of at least one of the magnetic probes is an active electrode of a bipolar electrode pair and the electrode of at least one of the other magnetic probes is a return electrode of a bipolar electrode pair. The magnetic probes are operatively connected to the driving device, which supplies radio frequency energy to the active electrode sufficient for a user to create transmural lesions therewith in the walls of an anatomical structure, which may be clamped between the magnetic probes.

In one aspect of the present invention, a magnetic probe is provided which includes a magnetic component and a camera. The camera is generally capable of providing at still and/or video images of a site, e.g., a surgical site. The magnetic component enables a user to navigate the camera through a subject's body with an external magnet, such as a handheld magnet or stereotaxis system.

In one aspect of the present invention, a magnetic probe is provided which includes a magnetic component and a biopsy element. The biopsy element is generally capable of taking a biopsy sample of tissue in a structure having a lumen and the magnetic component enables a user to navigate the probe into the structure with an external magnet.

In one aspect of the present invention, method for treading atrial fibrillation is provided which includes the steps of inserting a first magnetic probe into a subject's heart, clamping a wall of the subject's heart between the first magnetic probe and a second magnetic probe, applying energy to the wall of the subject's heart clamped between the magnetic probes sufficient to create a lesion therein, and removing the first magnetic probe from the subject's heart. In one embodiment, the first magnetic probe is an unattached magnetic probe and inserting the first magnetic probe into the subject's heart includes the steps of clamping an atrium of the subject's heart to prevent blood flow there through, incising the atrium, inserting the first magnetic probe into the atrium through the incision, closing the incision, and releasing the clamped atrium. The method of removing the first magnetic probe from the subject's heart may include the steps of clamping the atrium, opening the incision, withdrawing the first magnetic probe through the incision, closing the incision, and releasing the clamped atrium.

Additional aspects of the present invention will be apparent in view of the description that follows.

### BRIEF DESCRIPTION OF THE FIGURES

FIG. 1 is a cross sectional view of a heart showing anatomical features thereof;

FIG. 2 depicts a magnetic surgical instrument system according to various embodiment of the present invention;

FIG. 3 is a cross section view of a heart showing a pair of magnetic probe introduced thereto to clamp the ventricular wall of the heart, according to one embodiment of the invention;

FIG. 4 is a cross sectional view of a stomach showing a pair of magnetic probes introduced thereto to clamp the wall of the stomach, according to one embodiment of the invention;

FIG. 5 is a cross sectional view of a stomach showing a pair of magnetic probes introduced thereto to clamp the wall of the stomach, according to another embodiment of the invention; and

FIG. 6 is a cross sectional view of a stomach showing a pair of magnetic probes introduced thereto to clamp the wall of the stomach, according to another embodiment of the invention.

### DETAILED DESCRIPTION OF THE INVENTION

The surgical instrument systems of the present invention provide, among other aspects, the ability to perform less invasive transmural surgical operations. Although the present invention may be described by way of example in relation to treating certain types of diseases and also applicable to certain types of organs, it is understood by those skilled in the art that the present invention is not limited thereto in either respect. It is therefore understood that the present invention may be applied to treat any type of disease and anatomical structures that may benefit from less invasive transmural surgical operations as described herein.

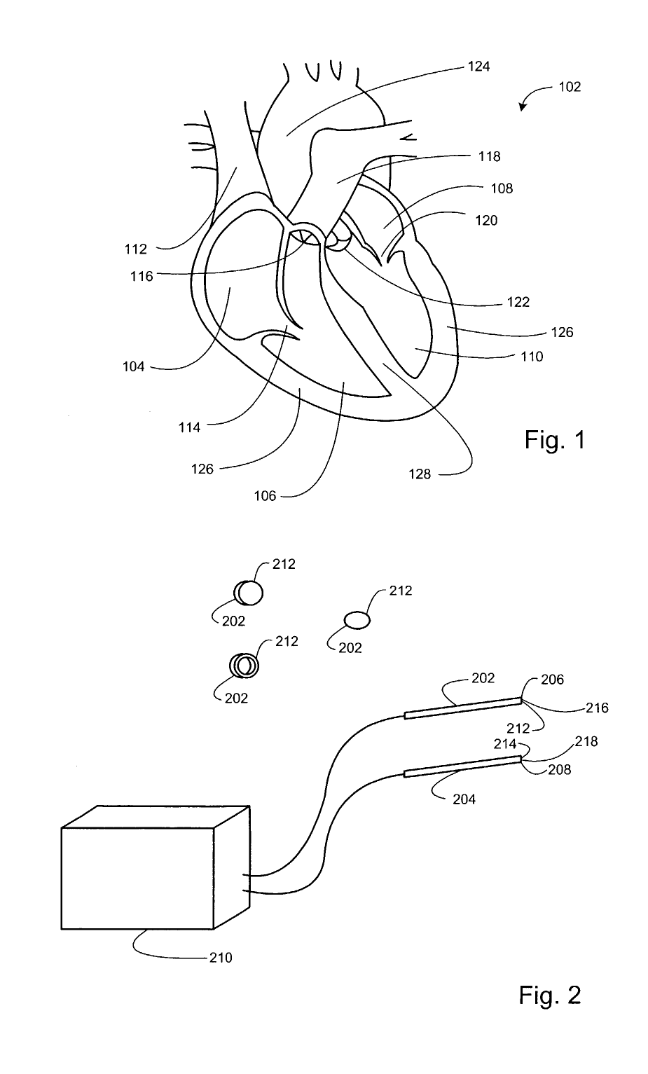

Referring to FIG. 1, a human heart 102 is generally composed of four chambers, the right atrium 104, the right ventricle 106, the left atrium 108, and the left ventricle 110. The superior vena cava 112 and inferior vena cava feed blood to the right atrium 104. The right atrium 104 contracts and thereby sends blood through the tricuspid valve 114 into the right ventricle 106, which then contracts to send blood though the pulmonary valve 116 into the pulmonary trunk 118. The pulmonary trunk 118 feeds the pulmonary arteries, which terminate in the lungs. Blood oxygenated by the lungs returns into the left atrium 108 through the pulmonary veins. The left atrium 108 similarly contracts to send oxygenated blood through the mitral valve 120 into the left ventricle 110, which will subsequently contract to send the blood through the aortic valve 122 into the aorta 124. The aorta 124 feeds oxygenated blood to the arteries of the rest of the body. The left and right ventricles are separated by the interventricular septum 128 and the heart is delineated at least in part by the ventricular sidewalls 126.

Referring to FIG. 2, a magnetic surgical instrument system for use in transmural surgical operations, according to one embodiment, includes at least two magnetic probes, e.g., a first magnetic probe 202 and a second magnetic probe 204, each of which includes components that provide the functional aspects of the invention. A probe is herein used to generally denote an instrument that may be introduced into a body cavity or organ, including, but not limited to catheters, wands, spheres, cylindrical rings, pills, etc. A transmural surgical operation is herein used to denote a surgical procedure that involves compressing or passing at least partially through the wall of anatomical structure having a lumen. A transmural surgical operation includes, but not limited to, clamping, creating lesions, cauterizing, injecting substances, stapling, suturing, and cutting the walls of the anatomical structures.

In one embodiment, at least one of the magnetic probes 202, 204 is a slender instrument, such as a catheter or wand, which includes a distal end 206, 208 wherein the components providing the functional aspects of the invention are located. Accordingly, the distal ends 206, 208 of the slender magnetic probes will include therein components that provide the functional aspects, e.g., functional components 216, 218, for which the magnetic surgical instrument system is adopted. Thus, where the surgical instrument is adopted to cauterize or create lesions in tissue, e.g., via radiofrequency (“RF”) radiation, at least one of the magnetic probes 202, 204 will be operatively connected to a driving device 210 and will include an RF electrode or electrodes, either monopolar or bipolar, at the distal end 206, 208. Similarly, where the surgical, instrument is adopted for microwave, laser, or cryothermic cauterization or ablation, the distal end 206, 208 will include components to create lesions via microwave, laser, and cryothermic energy respectively. The magnetic probes 202, 204 may be used to only provide clamping pressure in which instance the functional components are magnetic components 212, 214. In one embodiment, two of the magnetic probes 202, 204 are slender instruments each of which include distal ends 206, 208 having functional components 216, 218 therein.

The magnetic probes 202, 204 each include corresponding magnetic components 212, 214 therein, which allow a user to clamp the walls of the anatomical structure with the magnetic forces provided by the corresponding magnetic components 212, 214. The magnetic components 212, 214 may be permanent magnets, electromagnets, a metal attracted to a magnet, or a combination thereof. In one embodiment, one of the probes is spherical or oblong, such as in the shape of a pill, with the magnetic component 212 comprising a permanent magnet or a metal attracted to a magnet so that the probe may be unattached with respect to the driving device 210. This facilitates, for example, using multiple unattached probes in a single surgical intervention. The unattached probes, as well as attached probes, after being introduced into the patient's body may be navigated to the site of the surgical procedure with the assistance of an external magnetic field, such as with a handheld magnet or a magnetic stereotaxis system.

In one embodiment, the magnetic component of at least one slender magnetic probe 202, 204 is an electromagnet that may be selectively energized thereby allowing the magnetic clamping force between the magnetic components 212, 214 to be turned on and off. In another embodiment, at least one of the magnetic components 212, 214 is an electromagnet and the driving device 210 is adopted to provide variable and/or adjustable magnetic clamping force with the magnetic probes 202, 208. The variable and/or adjustable clamping force feature may be accomplished with circuitry, which provides for increasing and decreasing the amount of magnetic clamping force produced by at least one of the electromagnets. The magnetic clamping force produced by electromagnets may be varied, for instance, by varying the amount of current supplied to at least one electromagnet or by varying the number of energized coils associated with the electromagnet. It is understood that the magnetic components 212, 214 may be configured to produce various levels of clamping pressure, however, it is preferred that magnetic components produce sufficient magnetic clamping pressure to clamp or tightly sandwich the walls of the anatomic structure, e.g., the cardiac tissue, between the distal ends 206, 208 so that a transmural surgical operation may be performed thereon in a precise, complete, and minimally invasive manner as described herein. It is understood that the amount of pressure necessary to clamp the walls of the anatomical structure varies depending on the type of tissue, e.g., striated cardiac muscle tissue vs. smooth muscle tissue, and the relative strength of the tissue. For instance, the pressure that may be applied to tissue may vary between 10 to 100 psi, whereas the pressure applied to cardiac muscle may be in the higher end of the spectrum without crushing the tissue and whereas the pressure applied to softer tissue may be in the lower end of the spectrum to prevent crushing of the tissue. The magnetic clamping force, for example, may also be sufficient to drive a staple, blade, or needle into the wall of the anatomical structure.

The driving device 210 to which the magnetic probes 202, 204 may be operatively connected generally provides the driving signal enabling the functionality of the magnetic probes 202, 204. Accordingly, the driving device 210 that enables the functionality of the magnetic probes may vary according to the functionality for which the magnetic surgical instrument system is adopted. For instance, where at least one of the magnetic components 212, 214 is an electromagnet, the driving device 210 includes therein circuitry to provide electric current to the electromagnet to produce the necessary magnetic clamping force to attract the magnetic probes 202, 204 and where the magnetic probes are slender instruments, to attract the distal ends 206, 208 of the magnetic probes 206, 208. Where the surgical instrument is adopted to provide variable and/or adjustable clamping pressure between the distal ends 206, 208, the driving device 210 includes circuitry allowing a user to vary the magnetic clamping force produced by the electromagnet. The driving device may also be adopted to receive data during the transmural surgical operation and display the data or act on the data accordingly. In one embodiment, for instance, at least one of the distal ends 206, 208 of the magnetic probes 202, 204 include therein a pressure sensor, which supplies data associated with the amount of the magnetic clamping force produced or the amount of pressure applied with the magnetic probes 202, 204. The data may be used to monitor and/or limit the pressure applied to the anatomical structure wall. The driving device 210 may actively monitor the pressure and adjust the clamping pressure accordingly by either increasing or decreasing the magnetic attraction between the distal ends 206, 208.

As noted above, the magnetic surgical instrument system may be adopted to provide various types of functionality with respect to transmural surgical operations. In one embodiment, the magnetic surgical instrument system is adopted to produce transmural lesions in the tissue of the anatomical structure wall. This may be accomplished, for instance, with corresponding functional components 216, 218 on each of the distal ends 206, 208, which generally allows a user of the device to create lesions on one side or on each side of the anatomical structure wall. In so doing, the present invention allows a user to create complete transmural lesions through the anatomical structure wall with less energy, applied to one side or each side of the anatomical structure wall, than would otherwise be required to produce complete transmural lesions, thereby limiting unnecessary excess charring of the anatomical structure tissue. Moreover, the ability to clamp or compress the anatomical structure walls between the distal ends 206, 208 of the probes 202, 204, stabilizes the tissue in relation to the distal ends 206, 208, and also imparts low resistance to the tissue thereby further providing the ability to create precise low energy transmural lesions. In one embodiment, only one of the magnetic probes 204 includes a functional component to allow a user to create a lesion therewith. Unlike other ablation catheters, the ability of the magnetic component to clamp or compress the tissue there between provides the ability to create precise lower energy transmural lesions, which limits excess charring of the anatomical structure tissue.

Where the functional components 216, 218 provide RF radiation, or other types of energy, such as microwave, laser, cryothermic, etc., to create the transmural lesions, at least one of the magnetic probes 202, 204 includes at least one electrode, or equivalent component based on the type of energy provided, at each of the distal ends 206, 208. An electrode is used herein to denote a device capable of communicating energy, e.g., RF, microwave, laser, cryogenic energy, etc., to the tissue of the anatomical structure. In one embodiment, the surgical instrument system is adopted to cauterize or create lesions in tissue using bipolar RF energy in which instance the system includes at least two magnetic probe 202, 204, where one of the magnetic probes 202, 204 includes an active electrode of a bipolar electrode pair and at least one of the magnetic probes includes a return electrode of the bipolar electrode pair.

The amount of energy, e.g., power and frequency, that may be applied to the anatomical structure tissue via the electrode or equivalent structure will vary depending on factors, such as the thickness of the anatomical structure walls, the temperature of the anatomical structure, the desired degree of transmural penetration, etc. Thus, the driving device 210 includes therein circuitry such that energy supplied by it to the electrodes, or equivalent components may be provided in variable and/or adjustable amounts. Where RF energy is being used, the driving device 210 may be adopted to variably and/or adjustably provide about 10 to about 60 watts of energy at a frequency between about 100 KHz to about 1 MHz. With regard to microwave energy, the driving device 210 may be adopted to power ranging between about 25 to about 60 watts at a frequency of about 800 MHz to about 2 GHz. The power and/or frequency supplied to the electrodes or equivalent component may be selected or specified by the user.

In one embodiment, at least one of the distal ends 206, 208 of slender magnetic probes includes therein a temperature sensor that may be used to monitor the temperature at the site during the application of the energy thereby enabling the user or the driving device 210 to control and adjust the power and frequency settings to optimally produce the desired lesions. In one embodiment, each of the distal ends 206, 208 of a pair of slender magnetic probes 202, 204 includes therein a temperature sensor and the user or the driving device 210 is capable therewith to independently adjust the power supplied to each of the electrodes or equivalent components of the magnetic probes 202, 204. In one embodiment, the frequency and power to the electrodes is monitored and adjusted automatically by the driving device based on the temperature readings. In one embodiment, the driving device 210 includes therein timing circuitry, which measures or monitors the amount of time that energy is being applied to the walls of the anatomical structure. The driving device may adjust or cutoff the amount of energy supplied to the electrodes based on the energy timing. The driving device may also include circuitry to allow a user to select or adjust the amount of time for which the energy is being applied to the tissue.

The magnetic surgical instrument according to the present invention may also be adopted to provide numerous other functions with respect to transmural surgical operations. For instance, at least one of the distal ends 206, 208 of a slender magnetic probe 202, 204 may include therein functional components 216, 218 enabling a user to incise, inject, suture, and/or staple walls of the anatomical structure. In these instances, each of the magnetic probes 202, 204 includes corresponding components to perform the desired function. For example, where the surgical instrument is adopted to incise or cut tissue clamped between the magnetic probes 202, 204, at least one of the functional components 216, 218 is a blade or similar cutting instrument, thereby allowing a user to incise partially or completely through the tissue in a precise and minimally invasive manner. Similarly, where the surgical instrument is adopted to apply staples to the tissue clamped between the magnetic probes 202, 204, at least one of the functional components 216, 218 is a staple mechanism containing a staple and capable of driving a staple into the tissue. The blade and stapler mechanisms may be controlled in a variety of ways, such as pneumatically, electrically, mechanically, etc. In some embodiments, the driving force necessary to cut and staple is provided by the magnetic components. Thus, in these instances the magnetic clamping force used to clamp the anatomic structure wall also provide the force to cut and staple. In one embodiment, one of the probes includes a retractable stapler, which is capable of driving a circular or linear shaped staple into the anatomical wall. In this instance, the magnetic probe that is brought into contact with the outer side of the anatomical structure includes a stapling mechanism with a staple therein which is ejected out of the stapling mechanism upon the application of magnetic clamping force from the corresponding magnetic components onto the stapling mechanism at the distal tip of a slender magnetic probe. In one embodiment, the blade is retracted into one of the magnetic probes. In this instance, the magnetic clamping force applied to the blade mechanism at the distal tip of the magnetic probe from the magnetic components causes the blade to eject and thereby incise or ablate the tissue clamped between the magnetic components.

Where the instrument is adopted to inject substances into or through the tissue clamped between the distal ends 206, 208, at least one of the functional components 216, 218 includes means for injecting substances into the tissue, such as a needle or needles, precisely and in a minimally invasive manner. In one embodiment, at least one of the probes includes therein at least one needle retracted therein which ejects upon the application of force on the distal tips of the probes with the magnetic components. The substance to be injected therewith may be stored in a reservoir remotely or locally proximate to the distal end of the functional component which includes the needle to provide the substance to be injected the tissue thereto. Alternatively, the injection may be administered pneumatically wherein the substances are introduced into the tissue in a blast of air. At least one of the functional components 216, 218 may also be able to biolistically introduce coated particles, e.g., coated with genetic material, into the cells of the tissue clamped between the distal ends 206, 208 of the magnetic probes 202, 204, thereby enabling minimally invasive in-vivo gene therapy.

Where the instrument is adopted to suture tissue clamped between the distal ends 206, 208, at least one of the functional components 216, 218 includes at least one suturing needle and a suture receptacle, which provides the sutures for the suturing needle. This particular embodiment may advantageously be applied to repair the interventricular septum or to repair septal defects by suturing and/or attaching a prosthetic wall thereto, to suture folds of tissue, etc. In one embodiment, the suturing aspect enables a user to suture the tissue sandwiched between the magnetic probes similar to a sewing machine, such that the user may suture in linear and/or circular patterns. The instrument may also be adopted to thread a suture into the tissue from each side of the tissue thereby enabling the user to create a drawstring with the sutures that can be pulled to draw the tissue together to e.g., close a hole, or to draw tissue and a prosthetic attachment together, e.g., to block a hole.

The magnetic probes 202, 204 may be manufactured from a variety and/or a combination of biocompatible and non-biocompatible materials, such as polyester, Gortex, polytetrafluoroethyline (PTFE), polyethelene, polypropylene, polyurethane, silicon, steel, stainless steel, titanium, Nitinol, or other shape memory alloys, copper, silver, gold, platinum, Kevlar fiber, carbon fiber, etc. Where non-biocompatible materials may come into contact with the anatomic structure, the components made from the non-biocompatible materials may be covered or coated with a biocompatible material. In one embodiment, the magnetic probes are made in part of a flexible biocompatible polymer, which allows a user to navigate to the site of the transmural surgical operation though a patient's vasculature. This, for instance, may be useful for transmural surgical operations involving the interventricular septum 128. In another embodiment, at least one of the magnetic probes 202, 204 is made in part of an essentially rigid biocompatible polymer. A fairly rigid probe facilitates, for instance, accessing the exterior of a targeted anatomical structure, such as the heart, through an incision into the patient's thoracic or abdominal cavity.

The magnetic probes 202, 204 may also include various additional features to facilitate the transmural surgical operation. For example, at least one of the magnetic probes 202, 204 may be equipped with means for irrigating and aspirating the surgical site. Additionally, the probes may include therein a camera that provides still or video images of the site. In one embodiment, the camera is included in an attached or unattached magnetic probe, such as in the shape of a pill, which may be navigated through a subject's body with an external magnet, such as a handheld magnet or a stereotaxis system. In another embodiment, an unattached magnetic probe is adopted to take a biopsy of tissue within a lumen. A biopsy element, such as a clamp or clippers, retractable or otherwise, at the distal end of the probe can be used to take the biopsy. For example, the probe can be dropped into the lungs and controlled via an external magnetic navigate the probe to a specific location. This provides an advantage over traditional bronchoscopies, which require a bronchoscope and insertion into the lungs by threading a catheter like scope down the lungs. The magnetic surgical instrument system, according to the present invention, may be applied to treat various diseases, including, but not limited to, ablating arrhythmias, tumors, etc., in various anatomical structures having a lumen. In one embodiment, at least one of the magnetic probes is shaped like a tubular ring to which an end of a graft may be attached and navigated to the particular anatomical structure to facilitate an anastomosis of the graft to the anatomical structure.

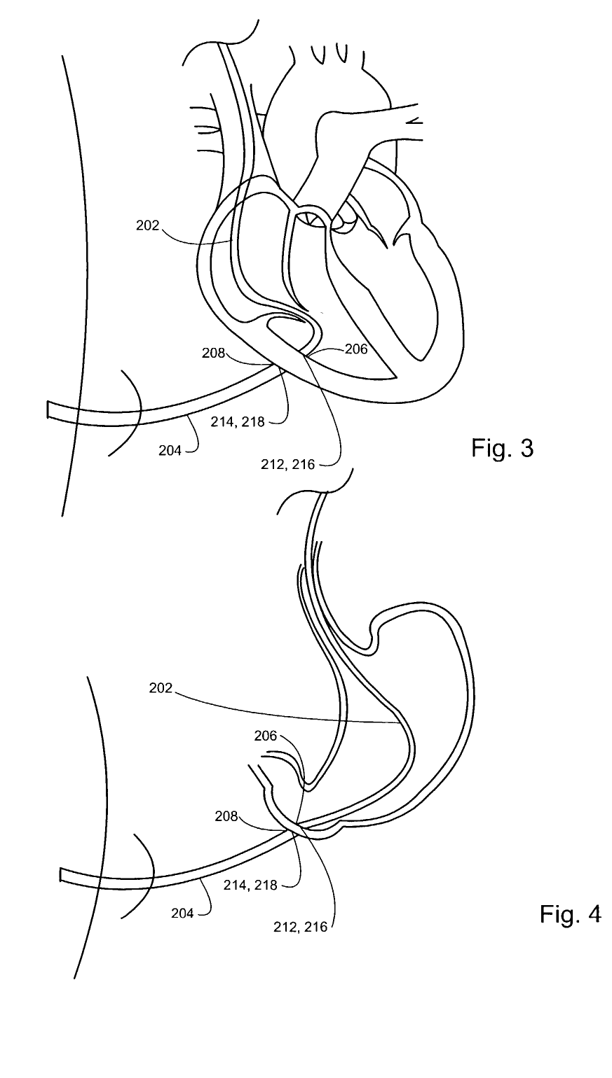

In one embodiment, the magnetic surgical instrument system is used by introducing one of the magnetic probes 202, 204 into the lumen of the anatomical structure and another magnetic probe introduced into a bodily cavity to access the exterior surface of the anatomical structure, thereby allowing a user to clamp a wall of the anatomical structure between the magnetic probes. Referring to FIG. 3, the magnetic surgical instrument system, according to one embodiment, is used to treat various disorders associated with the heart, such as arrhythmias. In this instance the first magnetic probe 202 may be introduced into the relevant chamber of the heart, such as the right or left ventricle, intravascularly though either the super vena cave or the aorta thereby providing access to the inner surface of the heart, and the second magnetic probe 204 introduced through an incision into the thoracic cavity thereby providing access to the exterior surface of the heart. The magnetic components 212, 214 at the distal ends 206, 208 provide the magnetic clamping force to attract and bring the distal ends 206, 208 together to effectively clamp the ventricular sidewall between the distal ends 206, 208. As shown, the first distal end 206 comes into contact with the endocardium and the second distal end 208 contacts the epicardium, thereby enabling minimally invasive transmural surgical operations therewith, such as creating lesions, clamping, incising, stapling, injecting, etc. Although FIG. 3 depicts the surgical instrument with regard to the ventricular sidewall, the present invention may be applied to clamp any wall of the heart, such as the interventricular septum, interatrial septum, atrial sidewalls, vasculature, etc. In one embodiment, the magnetic clamping force may be alternately turned on and off thereby allowing a user to turn on the magnetic clamping force to perform the transmural surgical operation at one location and subsequently turn the clamping force off to facilitate maneuvering the probes to a second location and subsequent locations, such as may be required to perform the Maze procedure to cure atrial fibrillation. In one embodiment, the driving device 210 may be adopted to allow a user to continuously use the functional aspects, e.g., cut, ablate, staple, suture, etc., while maneuvering the magnetic probes along the anatomic structure. In this instance, it may be beneficial to provide the user with means to alternately turn the clamping force on and off, and to use the functional aspects of the magnetic probes independently from the clamping force. Herewith, the magnetic probes of the present invention advantageously enables users to ablate/cut in whatever pattern they choose with greater control, much like drawing with a pen, rather that the linear ablation/incisions capable with presently available ablation catheters.

In one embodiment, the probes are inserted into the ventricles during cardiac operations, for example to tread atrial fibrillation, by clamping the left atrium and/or the right atrium to prevent blood flow there through. The left and/or right atrium may then be incised, and the magnetic probe inserted into the incision. In the instance the magnetic probe is an unattached magnetic probe, such as in the shape of a pill, the incision may be closed, e.g., with staples, after the magnetic probe is inserted into the atrium. The magnetic probe within the heart may then move freely within the heart and to left or right ventricle, and navigated to the particular area of the heart that will be sandwiched between the probes for ablation with a second magnetic probe. The probes may be removed from the subject by withdrawing the probe from the ventricle into the atrium, clamping off blood flow to atrium, and stapling the incision in the atrium after the removal of the probe from the heart.

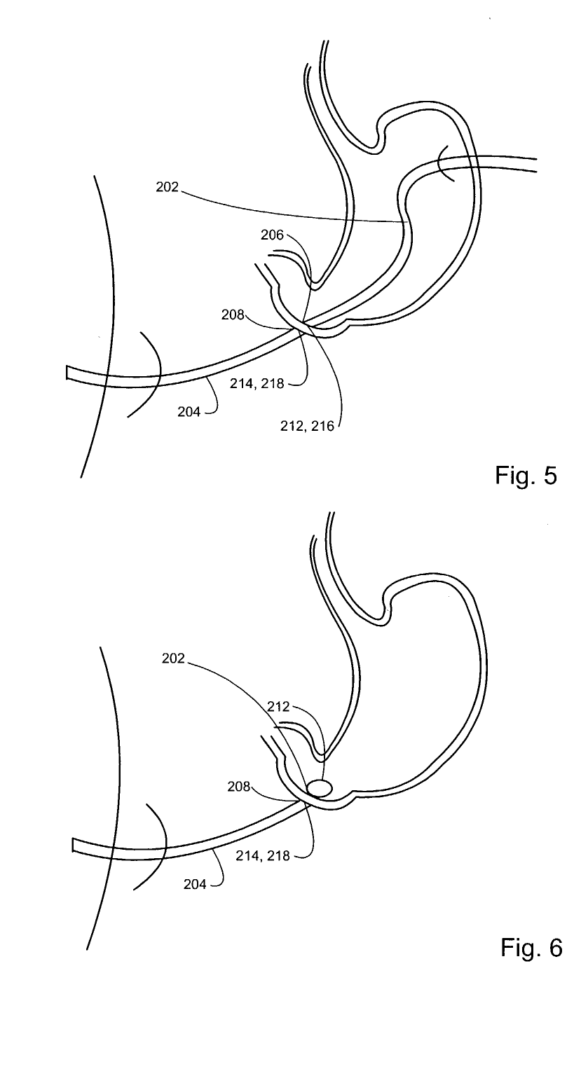

The magnetic surgical instrument, according to the present invention, may also be applied to perform transmural surgical operations in various anatomical structures, such as the stomach, as shown in FIGS. 4, 5, and 6, lungs, bladder, esophagus, intestines, thoracic cavity, abdominal cavity, blood vessels, etc. In certain instances, access to the lumen of the anatomical structure may be obtained by creating an incision in the anatomical structure sufficient to allow one of the probes to be inserted therein, as shown in FIG. 5. Referring to FIG. 6, at least one of the magnetic probes 202 may be unattached from the driving device 210 which may be introduced into the lumen of an anatomical structure, such as the stomach, and a slender magnetic probe 204 may introduced into the bodily cavity, such as the abdominal cavity, to access the exterior of the anatomical structure. The magnetic probes 202, 204 may then be brought together with the attractive magnetic clamping forces between the probes such that the wall of the anatomical structure may be clamped therewith. The magnetic surgical instrument may be used manually wherein a user, such as a surgeon, operates the instrument directly or in connection with automated means, such as with a surgical robot, wherein the user programs or operates the instrument remotely.

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A magnetic surgical instrument system for use in transmural surgical operations comprising at least two magnetic probes and a driving device, each magnetic probe having at least one functional component associated therewith comprising a magnetic component, wherein the magnetic component of a first magnetic probe comprises an electromagnet and wherein the first magnetic probe is operatively connected to the driving device which provides the electric current to energize the electromagnet and produce therewith a magnetic clamping force, the magnetic component for a second magnetic probe is a magnetic component selected from the group consisting of a permanent magnet, an electromagnet, and a metal attracted to a magnet, wherein the system enables a user to clamp walls of an anatomical structure between the magnetic probes.

2. The instrument of claim 1, wherein the driving device is adopted to enable a user to selectively energize the electromagnet of the first magnetic probe thereby allowing the magnetic clamping force between the magnetic components to be turned on and off.

3. The instrument of claim 1, wherein the driving device is adopted to provide an adjustable magnetic clamping force, thereby enabling a user to vary the pressure applied to the walls of the anatomical structure.

4. The instrument of claim 1, wherein the first magnetic probe comprises a pressure sensor which supplies data associated with the amount of clamping force produced with the magnetic probes, the data used to monitor the pressure applied to the wall of the anatomical structure.

5. The instrument of claim 4, wherein the driving device is adopted to actively monitor and adjust the pressure applied to the wall of the anatomical structure.

6. The instrument of claim 1, wherein at least one of the magnetic probes comprises a functional element comprising an electrode, the magnetic probe operatively connected to the driving device which supplies energy to the electrode sufficient for a user to create transmural lesions therewith in the walls of the anatomical structure clamped between the magnetic probes.

7. The instrument of claim 6, wherein the driving device is adopted to monitor the amount of time that energy is applied to the walls of the anatomic structure.

8. The instrument of claim 7, wherein driving device is adopted to adjust or cutoff the amount of energy supplied to the electrode based on the time that energy is applied to the walls of the anatomic structure.

9. The instrument of claim 6, wherein at least two of the magnetic probes comprise a functional element comprising an electrode, the magnetic probes operatively connected to the driving device which supplies energy to the electrodes sufficient for a user to create transmural lesions therewith in the walls of the anatomical structure clamped between the magnetic probes.

10. The instrument of claim 9, wherein the energy supplied to the electrodes comprises radio frequency energy and wherein one magnetic probe comprises an active electrode of a bipolar electrode pair and another magnetic probe comprises a return electrode of a bipolar electrode pair.

11. The instrument of claim 9, wherein the energy supplied to the electrodes comprises radio frequency energy and wherein the electrodes are one of monopolar or bipolar electrodes providing the user the ability to create transmural lesions from both sides of the walls of the anatomical structure.

12. The instrument of claim 9, wherein the energy supplied by the driving devices is selected from the group consisting of radiofrequency energy, microwave energy, laser energy, and cryothermic energy.

13. The instrument of claim 9, wherein the driving device is adopted to supply an adjustable amount of energy to the electrodes thereby enabling a user to vary the amount of energy to create the transmural lesions.

14. The instrument of claim 13, wherein at least one of the magnetic probes comprises a temperature sensor for monitoring and adjusting the temperature at the site of the transmural lesion.

15. The instrument of claim 14, wherein at least two of the magnetic probes comprise a temperature sensor and wherein the driving device is adopted to enable a user to independently adjust the amount of energy supplied to the electrode of each of the magnetic probes.

16. The instrument of claim 15, wherein the driving device is adopted to monitor the temperature at the site of the lesion and automatically adjust the energy supplied to each of the magnetic probes.

17. The instrument of claim 1, wherein the first magnetic probe comprises a functional element comprising a stapler mechanism containing a staple and capable of driving a staple into the wall of the anatomical structure clamped between the magnetic probes, the force necessary to drive the staple into the wall of the anatomical structure provided by the magnetic clamping force between the magnetic probes.

18. The instrument of claim 17, wherein the functional component comprises a retractable stapler containing a staple therein which is ejected out of the retractable stapler upon application of the magnetic clamping force on the magnetic probes.

19. The instrument of claim 1, wherein the first magnetic probe comprises a functional element comprising a blade capable of incising at lease partially through the wall of the anatomical structure clamped between the magnetic probes, the force necessary to incise the wall of the anatomical structure provided by the magnetic clamping force between the magnetic probes.

20. The instrument of claim 19, wherein the functional component comprises a retracted blade, which is ejected out of the magnetic probe upon application of the magnetic clamping force on the magnetic probes.

21. The instrument of claim 1, wherein the first magnetic probe comprises a functional element comprising at least one needle for injecting substances into the wall of the anatomical structure clamped between the magnetic probes, the force necessary to drive the needle into the wall of the anatomical structure provided by the magnetic clamping force between the magnetic probes.

22. The instrument of claim 21, wherein the functional component comprises a retracted needle, which is ejected out of the magnetic probe upon application of the magnetic clamping force on the magnetic probes.

23. The instrument of claim 1, wherein the first magnetic probe comprises a functional element comprising at least one suturing needle and a suture receptacle which provides sutures for the suturing needle, thereby allowing a user to suture walls of an anatomical structure clamped between the magnetic probes.

24. A magnetic surgical instrument system for use in transmural surgical operations comprising at least two magnetic probes and a driving device, each magnetic probe comprising a magnetic component and an electrode, wherein the magnetic component of a first magnetic probe comprises an electromagnet and the magnetic component of a second magnetic probe is a magnetic component selected from the group consisting of a permanent magnet, an electromagnet, and a metal attracted to a magnet, the magnetic probes operatively connected to the driving device which provides the electric current to energize the electromagnet and produce therewith a magnetic clamping force, and which supplies energy to the electrodes sufficient for a user to create transmural lesions therewith in the walls of an anatomical structure clamped between the magnetic probes.

25. A magnetic surgical instrument system for use in transmural surgical operations comprising at least two magnetic probes and a driving device, each magnetic probe comprising a magnetic component and an electrode, wherein the magnetic component of a first magnetic probe comprises an electromagnet and the magnetic component of a second magnetic probe is a magnetic component selected from the group consisting of a permanent magnet, an electromagnet, and a metal attracted to a magnet, the magnetic probes operatively connected to the driving device which provides the electric current to energize the electromagnet and produce therewith a magnetic clamping force, and which supplies radio frequency energy to the electrodes sufficient for a user to create transmural lesions therewith in the walls of an anatomical structure clamped between the magnetic probes from both sides of the wall of the anatomical structure, wherein the driving device is adopted to enable a user to selectively energize the electromagnet of the first magnetic probe thereby allowing the magnetic clamping force between the magnetic components to be turned on and off.

26. A magnetic surgical instrument system for use in transmural surgical operations comprising at least two magnetic probes and a driving device, each magnetic probe comprising a magnetic component and an electrode, wherein the magnetic components produce a magnetic clamping force which allows a user to clamp walls of an anatomical structure between the magnetic probes, and wherein the electrode of at least one of the magnetic probes comprises an active electrode of a bipolar electrode pair and the electrode of at least one of the other magnetic probes comprises a return electrode of a bipolar electrode pair, wherein the magnetic probes are operatively connected to the driving device which supplies radio frequency energy to the active electrode sufficient for a user to create transmural lesions therewith in the walls of an anatomical structure clamped between the magnetic probes.

27. A magnetic probe comprising a magnetic component and a camera, wherein the camera is capable of providing at least one of still images and video images of a site and the magnetic component enables a user to navigate the probe through a subject's body with an external magnet.

28. A magnetic probe comprising a magnetic component and a biopsy element, wherein the biopsy element is capable of taking a biopsy sample of tissue in a structure having a lumen and the magnetic component enables a user to navigate the probe into the structure with an external magnet.

29. A method for treating atrial fibrillation comprising:
inserting a first magnetic probe into a subject's heart;
clamping a wall of the subject's heart between the first magnetic probe and a second magnetic probe;
applying energy to the wall of the subject's heart clamped between the magnetic probes sufficient to create a lesion therein; and
removing the first magnetic probe from the subject's heart.

30. The method of claim 28, wherein the first magnetic probe is an unattached magnetic probe and wherein inserting the first magnetic probe into the subject's heart comprises:
clamping an atrium of the subject's heart to prevent blood flow there through;
incising the atrium;
inserting the first magnetic probe into the atrium through the incision;
closing the incision; and
releasing the clamped atrium.

31. The method of claim 29, wherein removing the first magnetic probe from the subject's heart comprises:
clamping the atrium;
opening the incision;
withdrawing the first magnetic probe through the incision;
closing the incision; and
releasing the clamped atrium.
